(19) United States
(12) Patent Application Publication
(10) Pub. No.: US 2015/0018902 A1
(43) Pub. Date: Jan. 15, 2015

(54) DEVICE AND METHOD FOR TREATMENT OF TINNITUS

(71) Applicant: Benny JOHANSSON, Malmo (SE)

(72) Inventor: Benny JOHANSSON, Malmo (SE)

(73) Assignee: AKLOMA TINNITUS AB, Malmo (SE)

(21) Appl. No.: 14/449,882

(22) Filed: Aug. 1, 2014

Related U.S. Application Data
(63) Continuation of application No. 12/544,873, filed on Aug. 20, 2009, now abandoned.
(60) Provisional application No. 61/155,322, filed on Feb. 25, 2009.

(30) Foreign Application Priority Data
Feb. 20, 2009 (RU) 2009106150

(51) Int. Cl. A61N 5/06

(52) U.S. Cl. CPC A61N 5/0613; A61N 2005/0657; A61N 2005/0662
USPC 607/95

(57) ABSTRACT

A device for the therapeutic treatment of tinnitus is disclosed, wherein a device for treatment of tinnitus comprising a topographic geometrical matrix and a support, wherein each topographic geometrical matrix is applied to the support and comprising
a) at least two concentric circles,
b) a circle having at least one circle inscribed, wherein said circle and said at least one circle have a common tangential arc point;
wherein for both a) and b) above the innermost circle is open or closed, the diameter of the outermost circle is at most 130 mm, and the diameter of the innermost circle is at most 3 mm, and wherein the ratio between the diameter of the outermost circle and the diameter of the next circle counted inwards toward the common center of the circles is at least 1.3; and
c) a set of identical circles arranged with the basis on the pattern of the flower of life shown in FIG. 2,
or asymmetrical variants of a)-c) having the ability to synchronize water according to the synchronization test as defined in Example 1,
as well as a method for therapeutic treatment of tinnitus, wherein at least one device as defined above and herein is applied on the body of a tinnitus patient, such as to the skin on the upper half of the body, such as to the head, such as in the vicinity of the affected ear, and is subjected to light with a wavelength of 360-4000 nm, such as daylight.

This application claims the benefit of priority of Russian Patent Application No. 2009106150, filed Feb. 20, 2009, and U.S. Provisional Application No. 61/155,322, filed Feb. 25, 2009. The contents of these applications are incorporated herein by reference.

### TECHNICAL FIELD OF THE INVENTION

The present disclosure relates to a device and to a method for treatment of tinnitus.

### BACKGROUND ART

Tinnitus is a common symptom and is defined as a sound sensation in the absence of an external and internal acoustical source or electrical stimulation (1). Tinnitus is caused by abnormal neural activity. There are many forms of tinnitus (2), i.e. it can be just noticeable, it can be an annoyance, it can reduce the quality of life by impairing the ability to carry out intellectual work or making it difficult to sleep, and it can be so severe that it leads to suicide. In clinical practice, tinnitus has been divided into three categories according to the way it is perceived (2): mild tinnitus, moderate tinnitus and severe (disabling) tinnitus. Mild forms rarely cause any problems; moderate tinnitus can interfere with intellectual work and sleep and often causes suffering. Severe tinnitus can have a major effect on a person's entire life, making sleep difficult and intellectual work impossible. The severity of tinnitus depends, however, on many factors, such as the level of stress, general health, sleep disturbance, and psychological or psychiatric conditions (1,8). There are no objective tests that can measure subjective tinnitus, and only the person affected can assess the level of severity.

In cross-sectional studies, tinnitus is reported to occur in approximately 10-15% of the general adult population and in 1-2% tinnitus is severe enough to cause a significant impairment of daily life (3,4,5). Hearing loss is often related to tinnitus, but tinnitus is also reported in people without a measurable hearing loss (3,4,5,6). In 7-year old children there was no correlation between the results from the hearing tests and the reported prevalence of tinnitus (7). The prevalence of tinnitus is known to increase with age (3,4,5) and at 55 years of age and above, the overall prevalence has been reported to be about 30% (7). Hearing difficulties are, in general, higher among men than women. There are many risk factors for tinnitus such as hearing loss, including age-related hearing loss and tinnitus may follow after exposure to noise, administration of certain drugs, infectious diseases and trauma to the auditory nerve (2).

Generally, it is agreed that subjective tinnitus is not a disease but a symptom of an underlying disorder, and the many forms of tinnitus probably have different pathophysiology (2). Most forms of tinnitus are abnormalities in the functioning of the central nervous system (CNS) and are often caused by expression of neural plasticity which may be brought about by abnormal input of sounds from the ear or through abnormal function of the auditory nerve, or by unknown causes (2). Other forms of tinnitus are associated with injuries of the sensory cells or auditory nerve fibres, thus creating the basis for hypersensitivity and hyperactivity (2).

The fact that tinnitus can occur after destruction of the auditory nerve provides strong evidence that tinnitus can occur without involvement of the ear and that the anatomical site of the physiological abnormalities that cause the sensation of tinnitus is the central nervous system (2). It also means that most forms of tinnitus are not generated at the location where the symptoms are felt (the ear) but rather that the symptoms are the result of phenomena in the CNS, similar to, for example, phantom pain. Several brain structures have been investigated and discussed with regard to their role in tinnitus (2). The neural activity that produces the sensation of tinnitus differs between the different forms of tinnitus and it is hypothesized that the sensation may be generated in neural structures that are not normally activated in processing auditory stimulus. As such, hyperactivity and/or abnormal signal processing in specific brain structures may cause tinnitus. It has been found that electrical or magnetic stimulation of the cerebral cortex and electrical stimulation of the skin around the outer ear can affect and modulate tinnitus.

Tinnitus is often accompanied by abnormal perception of sounds and many people have a lowered tolerance to sounds. Individuals suffering from tinnitus may experience an interaction with other sensory modalities, such as with the somatosensory system, e.g. via stimulation of skin or muscles. Tinnitus associated with muscular disorders can be resolved and the tinnitus usually decreases or disappears.

The finding that the perception of tinnitus is altered in some individuals with severe tinnitus by stimulation of the somatosensory system is a sign of involvement of non-classical auditory pathways. Neurons in the non-classical auditory pathways respond to more than one sensory modality while neurons in the classical pathways only respond to auditory stimuli. If input from other senses can modulate the perception of sound, it is taken as an Indication of involvement of the non-classical auditory system. For instance, it has been shown in experiments that electrical stimulation of the upper part of the body is more efficient than stimulation of the lower body.

The fact that most forms of tinnitus are disorders of the CNS in interaction with environmental cues puts emphasis on neuroscience and how urban settings, sounds and electromagnetic fields induce physiological distress (2,9), whereas the nature has an involuntary and adaptive restorative capacity and influence on the CNS (10).

Therapies available to tinnitus patients include pharmacological, physio-therapeutical, behavioural therapy, and various device-based approaches including electrical stimulation of the skin, ear or central nervous system. These arrays of therapies are not effective. No existing treatment provides a cure, and few patients experience significant benefits from therapy. At best, current therapies may minimize tinnitus symptoms in subsets of patients.

Progress in treatment of tinnitus may come from basic scientific research regarding how regulative changes occur both in auditory and other sensory perception as well as in the autonomous nervous system (ANS). Progress in treatment may also come from serendipitous observations, and from clinical experience of treatment of other disorders when these patients also have tinnitus, or during recovering from unhealthy conditions induced by disturbing external stimuli. Many effective treatments of a wide order of disorders have been discovered in that way.

Among other treatments, sound stimulation of the ear and electrical stimulation of certain neurons of the brain and central nervous system, skin areas behind the ear and other locations on the body have been shown to modulate tinnitus in some individuals, probably influencing activity in the non-classical auditory pathways. It has also been shown that the sympathetic branch of the ANS is activated and may be involved in noise induced hearing loss and tinnitus, which is related to stress as indicated by the fact that cortisol reactivity to psychosocial stress was blunted in tinnitus sufferers (2).

Many forms of tinnitus have similarities with different forms of neuropathic pain (11), especially chronic central neuropathic pain, and are also associated with different affective symptoms, like anxiety, fear, depression and even suicide. There is considerable evidence that the symptoms and signs of some forms of tinnitus and central neuropathic pain are caused by changes in specific parts of the CNS and are caused by neural plasticity. Neuropathic pain and tinnitus are both considered phantom perceptions sharing a similar pathophysiology and clinical symptoms. Both neuropathic pain and tinnitus are considered to be the result of maladaptive plasticity, where neural structures deprived of sensory input begin to receive and process “phantom” sensory information. The hypothesis behind expression of neural plasticity is a form of adaptation based on natural selection, where cells or cell groups deprived of sensory input actively go and look for information in order to survive (12). The basic concept is that supplying the missing information directly to the deaffected area will suppress induced symptoms by preventing or reversing plasticity.

Studies have supported this hypothesis and actually shown relief of symptoms in patients treated with phantom sound (tinnitus) and phantom pain (neuropathic pain) by application of electrical or magnetic stimulation of auditory and somatosensory structures respectively (12). The results indicate that input-deprived synapses may sprout to adjacent non-deprived areas in an atternpt to survive in long-term reorganization. The hypothesis suggests that sprouting occurs between areas where neurons are tuned to similar frequencies, stimulated by external information, e.g. from auditory or electrical signals and magnetic fields or by nature cues.

This evolution-like self-organizing process for building of brain structures allows adaptation of the organism within its own Internal constraints to the environmental load or information that support functional restoration and development of somatosensory or auditory tissues. The neural structures have, in conjunction with the role of environment in the regulation of gene activity evolved a cooperative strategy for their survival.

According to the psycho evolutionary theory (PET) (13) and attention restoration theory (ART) (10) a growing literature attests to the beneficial effects of nature, nature cues and restorative environments on well-being and promoting recovery from stress and mental fatigue experienced from urban life. There is clear and strong empirical evidence for the preference for natural over constructed environments, and especially for the inherent preference of humans to be in contact with water. Contact with water has been shown to foster emotional contentment as well as both improved mood and concentration. These results support an involuntary adaptive function manifest in human environmental preferences that is considered an important restorative survival factor.

The PET model emphasizes emotions and dwells on how nature reduces stress reactions associated with threat and challenge. Stress is considered a set of physiological responses to any situation that threatens well-being. The set of responses includes negative emotions and various physiological indicators of increased autonomic arousal. Recovery from stress can occur in settings that evoke moderate levels of interest, pleasantness, and calm. In such settings, positive affect replaces negative affect, negative thoughts are inhibited, and autonomic arousal decreases. Features of the setting responsible for recovery include moderate depth and stimulus complexity, a focal point, and the presence of appropriate content such as vegetation and water.

The second major theoretical approach of nature restoration is ART, which focuses on directed attention, the kind of attention that requires mental effort and can be fatigued from overuse. Directed attention fatigue leads to the inability to focus and has several unfortunate consequences, including performance errors, inability to plan, social incivility, and Irritability. Nature environments that enable recovery from directed attention fatigue are known as restorative settings. ART proposes that an effective restorative setting should have all four of the following properties;

a) fascination, which includes either content or mental processes evoked by the setting that engage attention effortlessly, thus allowing fatigued directed attention to rest.

b) being away, which implies that the setting is physically or conceptionally different from the usual environment.

c) extent, which means that the setting should be sufficiently rich and coherent that it can engage the mind and promote exploration.

d) compatibility, which implies a good fit between inclination or purpose and the kind of activities supported by the setting.

There is also a distinction between hard and soft fascination. Hard fascination is very intense riveting the attention and leaving little room for reflection. On the other hand, soft fascination is moderate in Intensity, enough to hold attention, and still leaving room for reflection. Settings with soft fascination also include a very important aesthetic component, which can help offset any pain that may accompany reflection. Also the architecture or the geometry of the setting, notably according to proportions of golden section, has restorative potential.

Various aspects of ART have received substantial empirical support that the time spent in nature—even if only for a short duration—can offer substantial restorative benefits. ART clearly predicts that contact with nature should in general alleviate directed attention fatigue and thereby improve any kind of functioning that depends on directed attention, e.g. several aspects of well-being like autonomy, environmental mastery, personal growth, positive relations, purpose of life and self-acceptance provide high scores on several measures.

According to both PET and ART the environmentally induced restorative-preference for adaptive behavioural self-regulation is measured from improvements in mood states and accompanied by physiological indicators such as reduced blood pressure and heart rate, and lower levels of stress hormones. In sum, based on a functional account of environmental preference, it could be expected to obtain a positive relation between the involuntary preference for a particular environment or natural specific cues and that particular environment's potential to provide restoration from stress or mental fatigue.

To conclude, there is a clear need to reduce or eliminate the symptoms of the different kinds of tinnitus more efficiently than existing treatments and therapies.

### SUMMARY OF THE INVENTION

In one embodiment the present disclosure relates to a device for treatment of tinnitus, wherein said device comprises at least one topographic geometrical matrix and a support, wherein each topographic geometrical matrix is applied to the support and comprising
a) at least two concentric circles,
b) a circle having at least one circle inscribed, wherein said circle and said at least one circle have a common tangential arc point;
wherein for both a) and b) above the innermost circle is open or closed, the diameter of the outermost circle is at most 130 mm, and the diameter of the innermost circle is at most 3 mm, and wherein the ratio between the diameter of the outermost circle and the diameter of the next circle counted inwards toward the common center of the circles is at least 1.3; and
c) a set of identical circles arranged with the basis on the pattern of the flower of life shown in FIG. 2,
or asymmetrical variants of a)-c) having the ability to synchronize water according to the synchronization test as defined in Example 1.

Further, the present disclosure relates in another embodiment to a method for therapeutic treatment of tinnitus, wherein at least one device as defined above and herein is applied to the body of a tinnitus patient, such as to the skin of the upper half of the body, such as to the head, and such as in the vicinity of the affected ear, and is subjected to light with a wavelength of 360-4000 nm, such as daylight.

In a further embodiment, the present disclosure relates to a method for therapeutic treatment of tinnitus, wherein water or a water-containing medium which has been subjected to light that has passed a topographic geometrical matrix as defined above and thereby has been synchronized in the synchronization test as defined in Example 1 is administered orally to a tinnitus patient.

Further information about the present disclosure and the problems solved by it, as well as specific embodiments thereof, appears from the following description and the accompanying drawings, as well as from the appended claims.

### SHORT DESCRIPTION OF THE DRAWINGS

FIGS. 1a-1h and FIG. 2 show different examples of topographic geometrical matrices which are included in the device according to the present disclosure.

FIG. 3 shows a device according to the present disclosure applied behind the ear of a tinnitus patient.

FIG. 4 shows the spatial fractality of TGM-modulated light for an SS matrix at 634 nm.

FIG. 5 shows the result of measurements of the temperature at the freezing point for different matrixes.

FIGS. 6a-8c and 7 show results of measurements of the temperature at the freezing point for SS matrixes having different outer and inner circle diameters.

FIG. 8 shows the construction of one embodiment of the device according to the present disclosure.

### DETAILED DESCRIPTION OF THE INVENTION AND DIFFERENT EMBODIMENTS THEREOF

A device according to the present disclosure has been shown to be able to produce so called synchronized water by subjecting water or a water-containing medium to radiation with light within a certain wavelength range, wherein the light before it hits the water or the water-containing medium is caused to pass through the specifically designed topographic geometrical matrix present on the device. It has been found that said matrix due to its optimized design is capable of altering the properties of the passing light in such a way that when it thereafter hits the water or the water-containing medium, it creates a previously unknown synchronization of all the water molecules. The properties of the synchronized water differ from those of so-called clustered water (19-22) and similar types of water previously disclosed within the technical field.

The synchronized water produced shows unique physical properties in a water synchronization test in that it in a distilled condition at atmospheric pressure has shown a density of 0.997855 g/ml to 0.998511 g/ml at 22° C., a water temperature at the freezing point of −3.7° C. to −8.6° C., a melting point from 0.1° C. to 0.2° C., a surface tension (at 22° C.) from 72.3 dyn/cm to 72.4 dyn/cm, and a dielectric constant from 82.23 F/m to 82.77 F/m. These teachings are disclosed in detail in (14), and the synchronization test conditions and the results obtained for two different matrixes are disclosed in Example 1 below.

The parameters and the value ranges thereof listed above are indicative of when water or a water-containing medium is to be regarded as synchronized per definition, and thus of whether a specific topographic geometric matrix has the ability to reduce or eliminate tinnitus in a patient, as will be explained in detail below.

Thus, if a specific topographic geometrical matrix has the ability to synchronize water, said matrix thereby also has the ability to reduce or eliminate tinnitus in a patient when applied to the skin of said patient, whereby the water in the skin and in body fluids of the tinnitus patient is synchronized.

Further unique and specific features of synchronized water are that:

a) it shows a non-thermal magnetic oscillation frequency range of 4-50 μHz in a Faraday environment;

b) during exposure to daylight at room temperature for 10 h, it shows an average temperature increase of at most 0.1° C., while the corresponding average temperature increase for non-synchronized water is at least 0.5° C.; and

c) in relation to its original non-synchronized condition under otherwise identical conditions at the same time, it shows further specific properties, such as increased conductivity, a changed pH, a reduced redox potential, a reduced relative hydrogen, and a reduced dissipative geometrical entropy. These features and how they are measured are also discussed in detail in (14).

When the incident light from the light source is made to pass the topographic geometrical matrix, its character is changed in such a way that the geometrical entropy in the spectral electromagnetic light is changed in terms of its spatial form and field structure, an increased coordination of single wave components of both electric and magnetic nature leading to a “laser-like” coherent self-stabilizing light. These alterations may be measured on spectral light (specifically 634 nm) from an ordinary light bulb in a spectrophotometer emitting a non-coherent light. The alterations in the physical light properties after passage of the matrix may be registered with a highly sensitive video camera, whereupon the image is analyzed and evaluated mathematically by optical spectral imaging.

The expression “tinnitus” used throughout the present application is intended to mean any one of the three established categories of tinnitus, i.e. mild, moderate, and severe (disabling) tinnitus as defined in (2).

The expression “topographic geometrical” matrix used throughout the application text is intended to mean that the design of the matrix or the pattern it forms or constitutes is based on classical geometry created from interference between standing waves having fractal properties.

The expression “topographic” used throughout the application text is intended to mean a dynamic or variable geometrical form or structure in 2D or 3D format.

The expression “classical geometry” used throughout the application text is intended to mean a materialization in the form of physical geometric patterns created by interaction between standing wave phenomena of sound or light having different frequencies (sinus waves) in a medium, in which the vibration or the wave motion is manifested in structure and form (e.g. via a vibrating plate sprinkled with sand, wherein the sand, alternatively in spherical water droplets containing fine particles, self-regulatively creates standing waves based on the frequency applied and with a definable geometrical structure and form).

The term “standing wave” used throughout the application text is intended to mean a wave phenomenon produced by two wave motions moving in opposite directions and superposed on each other. Thereby bellies and nodes occur along the waves, as well as a wave which seems to be immobile, merely oscillating back and forth, i.e. a standing wave. The highest amplitude of the wave is present in the bellies and the smallest is present in the nodes, and the distance between the nodes is half a wavelength.

A standing wave in an air column is created, for instance, by reflecting a pressure wave forward and backward at the ends of a cavity. These ends then constitute nodes, and a standing wave is created between them. If energy is applied in a convenient way and at a convenient location, this process may be maintained in such a way that a resonance tone arises in the cavity, i.e. a resonant standing wave. The frequency of the tone is dependent on the distribution rate, which is a physical property of the medium in which the wave moves, and the distance between the nodes. Also overtones, multiples of the resonance tone, may be maintained by the same process.

The expression “fractal proportionality” used throughout the application text is intended to mean the presence of infinitely repeatable self-like structural elements, which by self-organization spontaneously create a geometrical structure and form (e.g. formation of planar ice crystals in nature).

The term “matrix” in the expression “topographic geometrical matrix” (TGM) is intended to mean an article or object which is to be subjected to the incident light and through which said light is to pass before hitting e.g. the body surface of the tinnitus patient to be treated, whereby the water molecules in the patients skin and in the body fluids are synchronized. The matrix is arranged on a support which is in direct contact with the body surface of the patient.

The above-mentioned matrix may in one embodiment be defined by its two-dimensional appearance in a plane which is perpendicular or mainly perpendicular to the radiation direction of the light. The expression “two-dimensional” appearance is here intended to more precisely mean the two-dimensional pattern that the matrix forms when viewed from the radiation source. Thus, in this embodiment the matrix may have a thickness or a depth which is very small in relation to its extension in the two-dimensional plane perpendicular to the radiation direction. In other embodiments the matrix may be defined by its three-dimensional appearance, such as in cases in which it constitutes a more pronounced three-dimensional geometrical object, e.g. when the above-mentioned thickness or depth is greater. In the practice of the present disclosure, the use of more pronounced three-dimensional matrixes is, so far, less useful than two-dimensional matrixes.

The support on which the matrix may be applied or arranged may be manufactured from any suitable material which does not influence the electromagnetic properties of the incident light passing through it. In one embodiment the support is transparent. The support may be made of glass, such as boron silicate glass (optical cover glass) or quartz glass (optical), plastic, cardboard, sheet metal, natural material or any other transparent material, such as laminates or foils.

The support for the matrix may have the form of a platform, a plate, a foil, etc. The matrix may be arranged on the support in any known way, e.g. in such a way that it has been plated, imprinted, glued, painted, taped, cast or laminated. In one embodiment the matrix has been imprinted on quartz glass or has been laminated. In one embodiment the support has the ability to adhere to human skin, and is preferably provided with an adhering surface.

In a highly useful embodiment for treatment of tinnitus, the device according to the present disclosure is a plaster on which the matrix has been imprinted and which is compatible with human skin. Such a plaster has an upper side provided with the topographic geometrical matrix and a bottom side having the ability to adhere to human skin. Any conventional adhesive surface or product providing an adhesive surface may be used. Said adhesive surface may be protected by a liner before use.

Several other plaster types and similar supports are also useful, as long as they do not Influence the electromagnetic properties of the incident light passing through them.

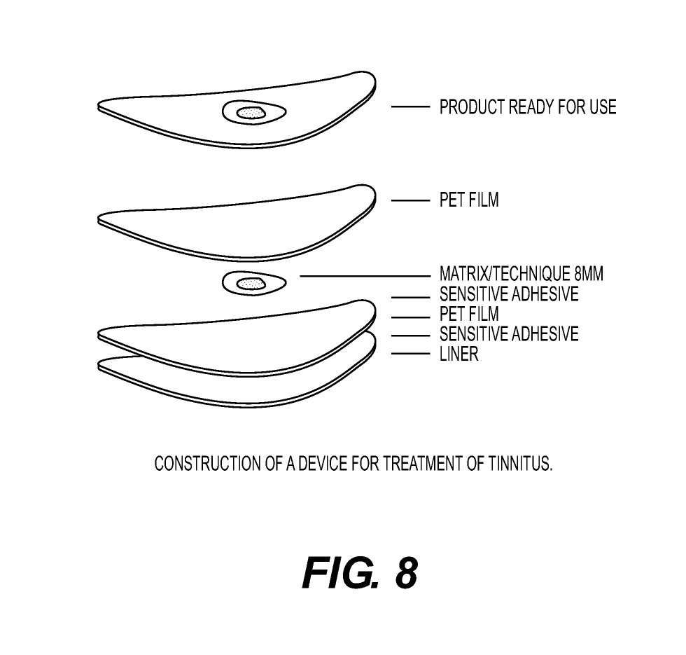

In FIG. 8 a specific embodiment of the device according to the present disclosure is shown. At the top a plaster product ready for use (Aklo®Tech Plaster) is shown. Below it is shown that the matrix, in this embodiment having an outer diameter of 8 mm, is arranged between two PET films and secured therebetween with an adhesive. Further, a liner is arranged on the bottom side of the lower PET film and is secured with an adhesive.

For the treatment of tinnitus, one or more devices according to the present disclosure, i.e. including the topographic geometrical matrix and the support, may be applied anywhere on the body, such as on the upper half of the body, such as on the head, such as in the vicinity of the affected ear. It is applied on a smooth skin part of the body with a view to promoting good adherence to the skin. In a specific embodiment said device is a plaster on which the matrix is arranged and is applied close to the ear showing tinnitus symptoms, e.g. at a distance of about 3-10 cm. The device should be arranged on a body part in such a way that the incident light hits the matrix in a substantially perpendicular direction in relation to the upper surface of the matrix. The plaster provided with the matrix should also be easy to attach and remove. It should be released from the skin after a few days' use due to hygienic reasons, but should be directly replaced with a new plaster.

The duration of the treatment period during the tinnitus treatment according to the present disclosure may vary due to the severity of the tinnitus symptoms, the type of tinnitus, and other patient characteristics. Normally, a treatment period of up to one month, preferably 2-3 weeks, is optimal, but satisfactory results have been obtained after both shorter and longer treatment periods, e.g. as short as a few hours. Should the tinnitus symptoms return or increase after a successful treatment, the treatment may be repeated, if necessary several times.

The device according to the present disclosure for treatment of tinnitus is subjected to light with a wavelength of 360-4000 nm, such as daylight, as radiation source. The surrounding conditions during treatment of a tinnitus patient, e.g. the surrounding temperature, the humidity of the atmosphere, etc, are of no relevance for the treatment. For optimal results, the patient should wear the device according to the present disclosure for treatment of tinnitus constantly, also during the night.

The size of the device according to the present disclosure for treatment of tinnitus may vary, but is during practice restricted by what is convenient for the patient. The plasters used in Example 2 below, for example, had an outer diameter of 27 mm (both the SS matrix and the “flower of life” matrix). Further several identical or different topographic geometrical matrixes may be applied to each plaster. The SS matrix embodiment used in Example 2 included a large circle with an outer diameter of 13 mm and a small circle with a diameter of 1 mm imprinted on the plaster used.

Devices according to the present disclosure on which the matrixes are extending in a substantially three-dimensional way, e.g. a cylinder, sphere or a part thereof, could possibly be used, but the best effects are obtained with matrices extending substantially in a two-dimensional way, i.e. which are as flat as possible with a minimal height. The colour of the plaster is not critical, and it may also have the same colour as skin, inter alia for cosmetic reasons.

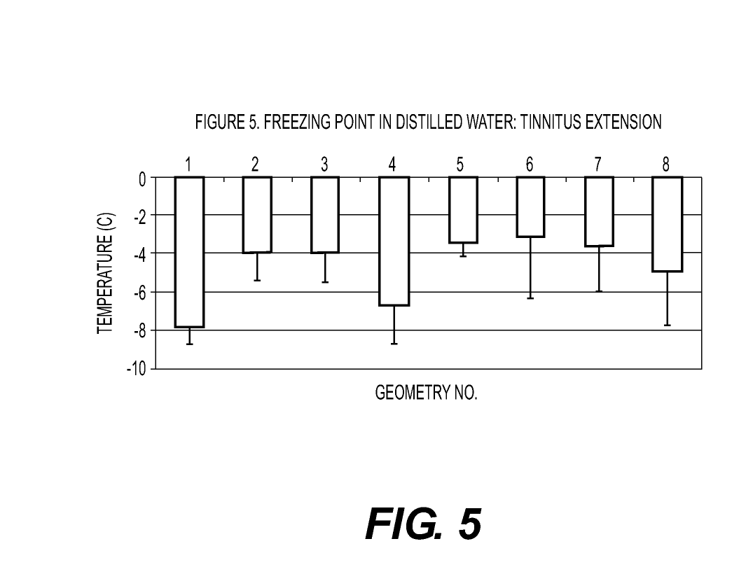

Further, when the device according to the present disclosure for the treatment of tinnitus is worn on the skin, it Is still effective if the incoming daylight is hindered by, for example, clothes or hair. The support as such, as mentioned above, does not essentially influence the electromagnetic properties of the incident light before the light hits the skin of a patient with a view to inducing a synchronization of the water in the skin and the body fluids, thereby eliminating or reducing the tinnitus symptoms. The form of the plaster is of no importance, although the form used in Example 2 is circular, and its main function is merely to support the matrix applied thereto and adhere it to the skin. In one embodiment, as shown in e.g. FIG. 4, the plaster is shaped like a banana with a view to better fitting the skin area along the back side of the ear. The plaster may also contain an UV coating.

The fields and lines present on or defining or constituting the matrix may also have a certain spectral colour or may be a metal foil for advantageous influence on the modification of the properties of the incident light. Convenient colours/metal foils for this purpose are gold, silver, copper, black, green, turquoise, red or other spectral colours. The reason why certain colours lead to better results is that a more precise fractal ordering is obtained.

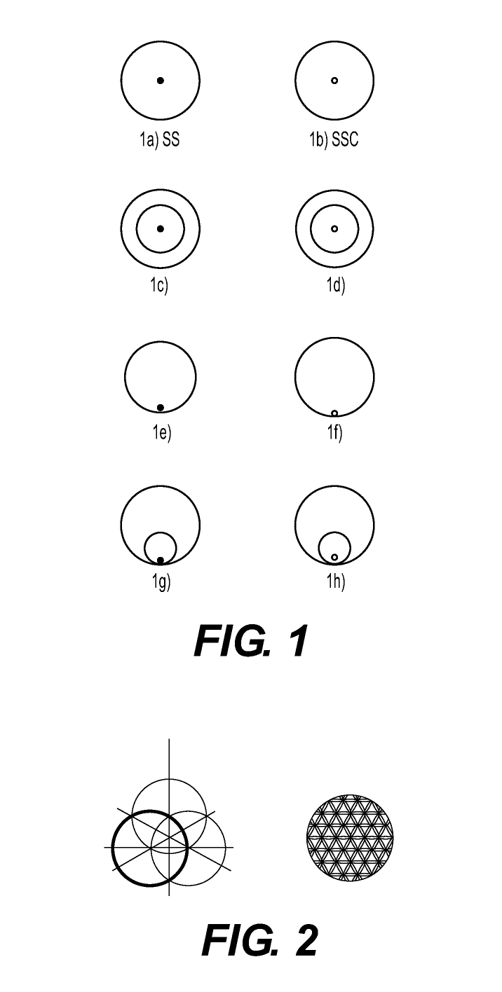

As stated above, the design of the topographic geometrical matrix according to the present disclosure, below sometimes called only “TGM” or “the matrix”, has a substantial influence on the modification of the properties of the incident light and thus provides the synchronization of the water, in the skin and body fluids of the tinnitus patient. Advantageous results are obtained in particular with geometrical matrix designs which are based on the geometry of the circle. Examples of effective matrixes are shown in FIGS. 1a-1h and 2.

The matrixes used in the device for treatment of tinnitus according to the present disclosure comprise
a) at least two concentric circles,
b) a circle having at least one circle inscribed, wherein said circle and said at least one circle have a common tangential arc point;
wherein for both a) and b) above the innermost circle is open or closed, the diameter of the outermost circle is at most 130 mm, and the diameter of the innermost circle is at most 3 mm, and wherein the ratio between the diameter of the outermost circle and the diameter of the next circle counted inwards toward the common center of the circles is at least 1.3; and
c) a set of identical circles arranged with the basis on the pattern o the flower of life shown in FIG. 2,
or asymmetrical variants of a)-c) having the ability to synchronize water according to the synchronization test as defined in Example 1.

The expression “outermost circle” used throughout the application text is intended to mean the largest circle in the matrix. The expression “innermost circle” use throughout the application text is intended to mean the smallest circle in the matrix.

The most simple embodiment of the matrix according to a) is a large circle concentrically enclosing a small circle (see FIGS. 1a and 1b). Other embodiments according to a) include a larger circle enclosing two or more concentric circles having a common center (see FIGS. 1c and 1d). Examples according to b), i.e. a circle having at least one circle inscribed, wherein said circle and said at least one circle have a common tangential are point, are shown in FIGS. 1e, 1f, 1g, and 1h.

The expression “closed circle” used throughout the application text is intended to mean that the incident light is not able to pass through the surface defined by said circle. Another embodiment, not shown in the Figures, includes a circle enclosing several smaller concentric circles, wherein one or more of the rings formed between the circles are closed. As to the embodiments disclosed above, the diameter of the outermost circle in the matrix is suitably at most 130 mm, such as between 2 and 130 mm. Devices containing matrix diameters larger than this are unpractical to use on the body. Tests with a diameter of the outermost circle of less than 2 mm give uncertain results due to measurement difficulties, but smaller diameters of the outermost circle, e.g. down to a nanometer level, should also be able to give the desired synchronization effect disclosed above.

The diameter of the innermost circle in the embodiments disclosed above is at most 3 mm, but varies suitably between 0.5 and 3 mm. Unsatisfactory results have so far been obtained with a diameter of more than about 3 mm, and smaller diameters than 0.5 mm give rise to measurement difficulties. However, smaller diameters of the innermost circle, e.g. down to a nanometer level, should also give the desired synchronization effect disclosed above.

The diameter of the circles discussed in the application text is calculated as a medium diameter of a circle having a certain line width, i.e. as half of the sum of the outer diameter and the inner diameter of said circle.

Moreover, for a) and b) above, the relationship between the diameter of the outermost circle and the diameter of the next circle counted inwards toward the common center of the circles is at least 1.3.

Particularly good synchronization and tinnitus treatment results have been obtained with specific ratios between the diameter of the outermost circle in the matrix and the next circle counted inwards toward the common center of the circles. These specific ratios have been found to be based on the well-known Fibonacci's sequence of numbers (23) (fₙ=θⁿ/5^{0.5} (0, 1, 1, 2, 3, 5, 8, 13, 21, 34, 55, 89, 144, 233 . . . ). In such a way that diameter ratios between any number being at least 2 in the sequence and any lower number than that being at least 1 in the sequence, including ±10% for each ratio, give a satisfactory result Thus, the two first numbers, i.e. 0 and the first 1, in the sequence are not involved when determining these ratios.

The ratio could also be expressed in the following way:

for all i>j,

1i
wherein

2j

i is chosen from 2, 3, 5, 8, 13, . . . and higher numbers in Fibonacci's sequence of numbers,

j is chosen from 1, 2, 3, 5, 8, 13, . . . and higher numbers in Fibonacci's sequence of numbers,

D1 is the diameter of the outermost circle in the matrix,

D2 is the diameter of the next circle counted inwards toward the common center of the circles.

More precisely, in at least some embodiments diameter ratios according to the definition above are:

2/1, 3/1, 3/2, 5/1, 5/2, 5/3, 8/1, 8/2, 8/3, 8/5 . . . etc, but also e.g. 55/8, 89/13, 233/144 etc.

The higher number of the sequence representing the diameter of the outermost circle when divided with the number just before said number in the sequence, the closer is the ratio to the limit θ=1,618 . . . i.e. the well-known golden ratio.

Due to the restricted outermost and innermost circle diameters disclosed above, a higher ratio than 233 is at present not of practical interest, but might be useful at shorter innermost diameters, e.g. down to nanometer level. The variation of ±10% for each ratio value means that satisfactory results are obtained also for slight variations of this diameter ratio. The lower diameter ratio limit of 1.3 defined above is established to approximately cover a 10% deviation downwards from the lowest ratio of 1.5 (3/2) based on the number sequence. Thus, the same ratio also apply between the diameter of the second outermost circle in the matrix and the next circle counted inwards toward the common center of the circles, in the case of a matrix having more than two concentric circles, but also in the case of non-concentric circles according to b) above. The above-mentioned ratio also applies to the perimeters of the above-mentioned circles.

The diameters of the circles in question may be measured by use of standard drawing software, e.g. Adobe Illustrater CS3.

The line widths of the circles discussed above are of no critical importance, but the line width of each circle ranges from 0.05 mm to 2.0 mm, such as from 0.1 mm to 1.5 mm, and such as from 0.5 mm to 1 mm. Different lines in one and the same topographic geometrical matrix may also have different widths, wherein the outermost circle in an SS matrix e.g. may have a line width of 0.5 mm and the innermost circle a line width of 0.1 mm. Generally, the larger the circle diameter, the larger the line width of said circle, and vice versa.

As stated above, one useful embodiment of the topographic geometrical matrix comprises a large open circle concentrically enclosing a small closed circle (see FIG. 1a, also designated SS). In the matrix shown in FIG. 1b (SSc), the small circle is not closed.

In a tested embodiment of the preferred SS matrix (see Example 1 and 2 below), the outer circle diameter is 13 mm and the closed inner circle diameter is 1 mm, said outer circle having a line width of 0.07 mm. Thus, the ratio between the outermost and the innermost diameters is 13, i.e. is included in the numbers of the sequence of preferred diameter ratio. In another embodiment the outer circle diameter is 55 mm and the inner circle diameter 8 mm, the outer circle having a line width of 2 mm. Both of these diameters are included in the Fibonacci's sequence of numbers, and the ratio between them is one of the preferred according to the definition above.

In another embodiment of the matrix two or more overlapping circles having identical diameters are present. Satisfactory tinnitus treatment results have been obtained with a matrix having such a pattern (see Example 2). Such an embodiment may be constructed starting from Vesica piscis, which is the intersection of two circles with the same radius, intersecting in such a way that the center of each circle lies on the circumference of the other, i.e. by first drawing a straight line starting from the center of a first circle (see the bottom to the left of FIG. 2). A second circle is then constructed to the right having its center on the same line, the arc of the second circle intersecting the center of the first circle. The center of a third circle (the upper circle to the left in FIG. 2) intersects the intersection of both first circles, and the arc thereof is tangent to the center of the first and the second circle, respectively. This construction scheme is then repeated until a larger pattern is obtained. The pattern shown to the right in FIG. 2 is the well-known “flower of life” pattern, constructed as disclosed above. This pattern may also be defined as a set of circles centered at hexagonal grid points, wherein the radius of each circle being equal to the grid point distance, wherein a total of four concentric circles are drawn at each grid point with radii of 1, 2, 3 and 4 times the grid point distance. The device according to the present disclosure comprises in one embodiment a matrix based on the flower of life pattern defined above and shown in FIG. 2. This matrix may also be regarded as a complex of identical SS matrixes in which the intersection of several circle arcs forms an Innermost closed circle in such matrixes.

This construction is also based on classical geometry and mathematical processes related to Fibonacci's sequence of numbers, as disclosed above. The amount of identical circles in this matrix embodiment is not critical and has no upper limit than what is practical to use on a support at the body of a tinnitus patient. The diameter of the whole matrix is governed by the amount of identical circles and is also not critical, but is normally at most 150 mm, such as at most 50 mm. The outermost diameter of each circle in the embodiment used in Example 2 is 13 mm, but may be more.

Thus, synchronization of water is obtained when the matrix in the substantially two-dimensional appearance contains the above-mentioned classical geometrical figures or patterns, in particular based on the geometry of the circle. However, the design of the matrix appearance may deviate slightly from the embodiments disclosed above, but the device according to the present disclosure also encompasses asymmetrical variants having the ability to synchronize water according to the synchronization test as disclosed in Example 1.

Thus, the circle on whose geometry the appearance of the matrixes is based may be, for example, slightly oval, and in other embodiments the circles do not have to be perfectly concentric and need not have an exact common geometrical center or tangential arc point. Circles of different sizes may also slightly overlap each other. Thus, all of the above described matrixes may deviate slightly from the above-mentioned forms a)-c). Another type of deviation could be one or more lines or patterns not based on the geometry of the circle. However, the deviation may only be such that the modification of the incident light for the synchronization of the water in the water-containing medium nevertheless takes place. Whether a certain matrix embodiment variant is effective for treatment of tinnitus may be established by performing the synchronization test disclosed in Example 1.

Thus, all topographic geometrical matrixes of the type disclosed above, and also deviations, variants and combinations thereof, are intended to be included in the scope of the present disclosure, as long as they have the ability to influence the incident light according to the definition in the synchronization test below, i.e. In such a way that the tinnitus symptoms of a patient are reduced or eliminated when the matrix is applied on the body of a patient via a support.

The topographic geometrical matrix according to the present disclosure is normally arranged in such a way that the incident light from the light source hits the present two-dimensional matrix perpendicularly to the radiation direction, but synchronization may also be obtained at a rotation angle of up to 180° (cf daylight).

The mechanisms behind the present disclosure will now be disclosed in more detail. As stated above, the present disclosure is based on a principle disclosed in (14), according to which ordinary daylight or spectral light is brought to pass through a transparent topographic geometrical matrix and thereafter is transferred to water, a medium containing water or a living organism, wherein the altered and highly organized qualities of light have the ability to influence the water so that it becomes synchronized, all individual water molecules being arranged at the same time in an self-identical (fractal) way to a stabilized homogeneous highly dynamic macrostructure and being thus provided with defined physical and chemical properties. Synchronized qualities are generated from a water state characterized by the ordering of water molecules that generally occurs next to hydrophilic macromolecular or solid surfaces (interfacial surfaces), where layers of ordered water are found with properties that differ from those of bulk water. The qualities of interfacial water have recently been identified in bulk water when it is exposed to very mild low-density energy (LDH) conditions, such as oscillating electromagnetic fields or even the influence of the Aklo® technology (14), the applicants own technology based on the present disclosure, etc. and transformed by means of water splitting into high-density energy (HDE) production. As disclosed above, the HDE water differs from ordinary water and exhibits unique physical properties in that it in a distilled condition has a higher density, lower freezing point, surface tension and dielectric constant. Other features are its higher heating capacity, changed conductivity, pH, reduced redox potential, relative hydrogen and dissipative entropy. Among the features of HDE water is that extended water clusters aggregating in an auto-stabilizing network can grow to macroscopic proportions and affect the condition of any external water in a liquid or gaseous state. The formation of HDE water leads to an increased concentration of reactive oxygen species (ROS) over time, or more specifically, the appearance of hydrogen peroxide as a result of the homolytic dissociation of water.

A functional food or drink could be defined by means of a scientifically proven beneficial effect on one or several target body functions that goes beyond normal nutritional effects and is related to either an improved state of health and well-being and/or disease risk reduction. A functional food is considered a normal product, which has an effect in amounts that can be consumed in an ordinary diet. The term functional water is employed to denote a water to be used in biological contexts in accordance with the definition of a functional food. The functional aspect creates conditions required for the self-regulating physiological mechanisms to re-establish homeostasis.

Preliminary studies on humans have shown that consumption of liquid (15, 16) or exposure to vaporous (17) water that has been conditioned using the present disclosure has a substantial physiological impact in terms of promoting restorative health. All three studies show that the ANS activity is shifted towards a resting parasympathetic response associated with reduced heart rate and increased heart rate variability (HRV) implicating restoration and maintenance of metabolic energy resources mediated by an involuntary adaptation to a conditioned water specific stimuli. The finding that exposure to natural stimuli containing conditioned vaporous water results not only in a reduction in an electromagnetic field (EMF)-induced (from a computer) physiological arousal and involuntary discrimination of Irrelevant external stimuli from the computer, but also in a condition of autonomic resonance and improved sympathetic/parasympathetic balance on the heart, contributes to important and new information, wherein the condition of liquid and vaporous water influences the processing of Information between nature cues and perceptive functioning supporting restoration, health and survival. A spectral frequency analysis identified a reinforced band in the frequency region around 0.1 Hz, which indicates that the exposure leads to an increased autonomic stability. These observations are consistent with the theories about PET and ART involving emotional and physiological stability, accompanied by sustained and increased levels of attention and perception.

The drinking of an average dietary volume of conditioned functional water promotes parasympathetic pathway activation in the CNS with a positive effect on antibody-mediated Immunity in saliva via Increased accessibility on mucosal surfaces, which protects against pathogenic invasion. The result also indicates normalization of the blood pressure in relation to a known blood pressure increase with ordinary water. The distinct difference in curve shape between the two waters may suggest that functional water has a stabilizing effect on normal blood pressure. Moreover, a significantly lower variation in the standard deviation of systolic blood pressure may indicate less diurnal changes in blood pressure variability. A self-regulative stabilization of blood pressure induced by functional water might have considerable predictive value for health outcomes, especially since an elevation in the variation of circadian blood pressure and hyper-amplitude-tension is associated with an increased risk of stroke and other vascular diseases. In fact, the increase in parasympathetic activity observed in relation to the ingestion of functional water may compensate for the previously reported increase in sympathetic activity in patients with hypertension.

In addition to the effects on short-term cardiovascular variables, consumption of functional water also positively impacts power spectral density parameters (PSD). The PSD analysis recalculates the frequency content of time domain data into three distinct frequency segments. Each frequency range represents different segments of the autonomic nervous system and provides information about frequency domain terms. Functional water consumption leads to a large increase in total power on the heart and a significant, increasing effect on five-minute oscillations of very low frequencies, indicating sympathetic activity. The increases in five-minute oscillations favour a shift to nonlinear sympathetic signals with self-similar fractal organization that supports a healthy condition in the heart rhythm (18). Elevated fractal scaling heart rate dynamics after intake of functional synchronized water defining a feature of healthy function adaptability indicated a higher capacity to respond to unpredictable stimuli and stresses, thus improving autonomic stability. According to classical concepts of physiologic control, healthy systems are self-regulated to reduce variability and maintain physiologic constancy. Contrary to the predictions of homeostasis the output of, for example, the normal human heartbeat fluctuates in a complex manner. Scaling properties suggest that the nonlinear regulatory systems are operating far from equilibrium, and that maintaining constancy is not the goal of physiologic control. Fractal analysis may provide new approaches to assessing human gate control in health and disease, as well as monitoring the aging process.

All living systems including humans are exposed to a huge variety of EMFs emanating from electrical power supply lines and various types of electrical equipment, for example, visual display terminals (VDTs), fluorescent lights, household appliances, cell phones and televisions, etc. The negative effects of EMFs have been extensively debated and investigated in recent years. Although no clear-cut causal relationship has been established between EMFs and physiological symptoms, several investigations have revealed serious health risks. An increasing number of people suffer from so-called electrical hypersensitivity (17). The most common complaints are related to cutaneous and neurological symptoms, e.g. headache, eye and general fatigue, and sensory symptoms such as itching, stinging, pain, burning sensations in facial skin and tinnitus or tinnitus-like symptoms, with a tendency towards increased persistency. Major physiological system symptoms, for example, the heart, central nervous system and respiratory disturbances, have also been reported. In severe cases, people cannot work in such environments or tolerate EMFs at all. Recently, electrical hypersensitivity has been categorized as a form of environmental intolerance and hyper responsiveness to stressor stimulation, with an underlying pathophysiological disregulation mechanism related to possible involvement of the autonomic nervous system.

In conclusion, our studies strongly indicate that the specific diameter ratios of the topographical geometric matrix of the plaster perceived by non-classical pathways act as a unique involuntary conditioned body water stimuli affecting the ANS. The improvement in sympathetic/parasympathetic balance and autonomic functional stability may increase synchronization in the oscillatory rhythmic patterns produced by different physiological systems. This synchronized physiological state emerged naturally in the presence of the conditioned vaporous water (17) and altered signals between the heart and the brain centers that control HRV and PSD, which it is hypothesized may affect cognitive, emotional and physiological systems. This effect can be attributed to the beneficial influence on information processing, resulting in higher efficiency and more harmonious functioning of the body, which can thus operate as an integrated system. The parasympathetic conditioning of the autonomic nervous system facilitated a pronounced adaptation to and tolerance towards EMF-induced stressor signals and tentative subsidence of tinnitus sensations. Hence, it may be hypothesized that these symptoms are caused by the incessant urban produced systemic EMF-load in sensitized subjects. Since improvements in HRV and PSD are significant Indicators of an immediate physiological response and behavioural adaptation, an increased tolerance to stress, recovery from distress, as well as maintenance and recharging of physical energy resources leading to a beneficial health outcome can be expected (15-17).

Natural stimuli are considered effortless, for instance a sea view or a scent of roses that makes our body relax involuntary and spontaneously. Natural stimuli also lower the heart rate. The same reaction is achieved through resting after letting the body work, for instance by walking. When resting, the heart rate is lowered as a result of lowering the body load. Urban stimuli such as traffic noise are highly stressful and require mental effort and attention and makes people fatigued from over-stimulation. Attaching the patch behind the ear makes our body react in the same way as it would in the presence of nature stimuli that can lower the heart rate.

When exposed to distressful environmental stimuli or when feeling anxious you may notice a rapid heartbeat, shallow, rapid breathing and tense muscles everywhere in the body. These physical reactions are the result of the involuntary “fight or flight” response system, an Ingenious resistive and defense mechanism. When a person senses something perceived as potentially threatening, a number of physiological changes take place in the body. The brain sends warning signals through the central nervous system (including the ear). The adrenal glands begin producing hormones, which cause the heart to beat faster and breathing to become more rapid. Muscles tense and pupils dilate. The person's body is getting ready to confront the threat and deal with it, or get far away from the threat as quickly as possible.

Some people have an early warning system that is a little too sensitive. It is exhausting and uncomfortable to spend time in a state of high alert. In addition, there are possible physical consequences to feeling stressed all the time, including high blood pressure, elevated resistance and tension, physical discomfort and pain. That is how the physical discomfort (muscle tension of the inner ear) of tinnitus sensations occurs.

When the resistance is taken away, it is easy for the body to relax and come down from this heightened state of alert. In tinnitus discomfort, it is helpful to discharge the load of physical energy to relieve muscular tension and pain by application of the device and method according to the present disclosure.

The duration of the synchronized condition of the water in the body fluid in the treated patient is permanent, unless the patient is subjected to outer disturbances, e.g. agitation, shaking or exposure to powerful electromagnetic fields. If the synchronized condition is not upheld due to any outer disturbance, a spontaneous return to the synchronized condition will soon take place.

When the body fluid in the treated patient is radiated in the method according to the present disclosure, the water molecules first hit by the incident light, i.e. In the skin, are synchronized. Thereafter, the synchronization is spread automatically to all water molecules in the body until all the water molecules in the body are synchronized. The synchronization effect takes place instantaneously and at the speed of light in both liquid and air.

The synchronized water obtained with the method according to the present disclosure is intended to mean, in addition to what has been defined above and in the appended claims, that two or more related events take place simultaneously in the water, wherein co-ordination between the oscillations in oscillators in the form of single water molecules takes place with spontaneous order and rhythm. Induction of synchronization in water means that the water assumes a condition of spontaneous self-similarity, in which each water molecule shows an oscillating condition which is identical to the condition of any other water molecule in the medium. The water shows molecular co-ordination and co-operativity and acts like a macroscopic infinite molecule, which reduces the entropy, increases the Gibb's free energy and delocalizes the access to surface-active electrons, which, as mentioned above, inter alia results in increased density, lower freezing point, higher melting point, increased dielectric constant, reduced surface tension, non-termic oscillation of pH, amended pH, increased conductivity, reduced redoxpotential and reduced relative hydrogen. A dissipative entropy change, followed by a temperature decrease, feeds back to a self-regulatory condition of a thermodynamic equilibrium. The phase shift in the water creates a coherent, well-ordered microscopic field, in which molecular integration reduces the normal electric resistivity of water and therefore increases the conductivity. It should also be noted that once synchronization takes place, it is complete, i.e. no partially synchronized water exists.

The difference between conventionally clustered water and synchronized water is disclosed in (14) and is the following: the clustered water in the meaning as it is known within the technical field is characterized by the presence of macroscopic permanently interactive water clusters having a limited voluminous size (LDV and HDV clusters, respectively) in a permanent exchange and reorganization of single water molecule's localization and migration as well as by instantaneous dissolution and re-formation of individual hydrogen bonds. Thus, clustered water is conventionally described from a classical dynamic perspective of chemical bonding.

According to the Swedish National Encyclopaedia (Vol. 19, 1996, pages 280-281), a cluster model is described as an instantaneous arrangement in water with a duration of about one nanosecond, wherein each water molecule in these clusters is bound to three to four other water molecules, while the molecules outside are non-bound. The arrangement and the boundary of the clusters are changing all the time by co-ordinated motion of the water molecules. However, the synchronized water obtained in the body with the method according to the present disclosure is to be regarded as a self-like co-operative system defined from an energetic perspective. The physical and chemical properties defined above unambiguously describe the presence of a coherent self-stabilizing synchronized water characterized by a stabilized geometry, dissipative properties, co-operative synergism and increased free energy, wherein the water acts as a macromolecular liquid or fluid crystal having uniform properties.

The synchronized water may be distinguished from normal, non-synchronized water in several ways which are disclosed in (14). The experiments performed in Example 1 show that synchronized water differs from other types of water, both in physical and in chemical aspect.

The synchronized water according to the present disclosure may, as also discussed above, also be used as a so-called functional water or a functional beverage or food for medical and health stimulating applications, such as for the prevention of ill-health via more efficient regulative homeodynamics, e.g. for stimulation of parasympatic activity and humoral immunity in connection with e.g. infections, for exhaustion problems and stress related problems, hypotonia and hypertonia, respectively, and for optimization of oxidative metabolism and energy utilization, as well as for reducing or eliminating tinnitus symptoms. A functional water or a functional food may be regarded as an effective food which during normal consumption consists of natural foodstuffs with documented physiological effect (24). This is related to diet factors influencing the human health condition with properties which may be measured objectively in the form of better opportunities for improved health and of an improved condition according to the subjective experience of the individual.

Recently, water has been considered a dissipative system (25), consisting of coherent water domains (CD's) (26) such that the CD's are able to oscillate and a coherence state can be established among them. The coherent oscillation involves all molecules in the system oscillating in unison in tune with a self-trapped electromagnetic field at a well-defined frequency. The coherent oscillations produce an ensemble of quasi-free electrons, able to collect low-density energy from the environment and transform it to high-density energy in the form of electron vortices. Since these vortices are cold because of coherence they cannot decay thermally, so their lifetime can be very long. The CD's includes a rotational frequency of the vortex. A change in the vorticity can be achieved by applying an internal or external electric or magnetic potential. An increase in the vorticity in the coherent water would induce a change in the amount of electrons leaked out from the CD's, which in turn would imply a change in physical and chemical parameters.

The high-density energy may activate biomolecules resonating with the water CD's. The presence of quasi-free electrons is a unique feature in CD liquid water and makes it different from all other liquids and produces a dissipative configuration (25). A dissipative configuration is an open system, able to exchange energy and entropy with the environment (26). In fact, the water CD's receives energy from the outside and uses it to charge the biomolecules that react chemically producing new molecule species and output energy. When a system changes from a well-defined initial state to a well-defined final state, the Gibbs free energy F equals the work exchanged by the system with its surroundings according to the equation: F=E−TS, where E is the energy of the system, T is the temperature and S the entropy (26). This formula shows that the equilibrium is the result of competition between energy and entropy. Temperature is what determines the relative weight of the two factors. At low temperatures, energy prevails with formation of a highly ordered low entropy system. At high temperatures the entropy is dominant and so is the molecular disorder.

The TGM can be seen as a diffraction device, comparable to a Fresnel zone plate (27), which organizes and focuses electromagnetic radiation such as light. We have analyzed the spatial distribution of light (at 634 nm) transmitted through TGM's of different geometrical configurations using a digital camera mounted inside a spectrophotometer. For the SS geometry, a stable TGM-induced increase in fractality of the light distribution was observed at ratios between the outer and inner circle diameter (O/I CD) above 5 (see FIG. 4). Thus, as long as the O/I CD ratio is larger than 5, a stabilized fractal organization of light will result from passing through the SS matrix. The most preferable O CD was 13 mm since the level of fractality in the O/I CD range of 7-13 was significantly more stable related to lower and higher O/I CD's.

As appears above, the most applicable geometry for high quality synchronization of water according to the present disclosure is based on two concentric circles, i.e. a big open circle with a closed circle in its center (SS matrix). This proportional geometry between the two circles strictly follows the Fibonacci's sequence of numbers. Opposed to Fresnel zone plates, which by a set of radially symmetric rings has the ability to focus ordinary day light by diffraction (27), the SS matrix makes up a spatial formation of “self-trapped” laser-like photons especially in the red part (634 nm) of the light spectrum (25). The SS geometry is a plausible representation of a standing wave in 2D format related to the standing wave resonance form or structure of molecular hydrogen. Recently, it has been brought to a physical insight that the 3D information of physical matter can be completely encoded in the geometric 2D form of the subject (28). Thus, the 2D highly organized standing wave SS template reconstructs a higher state of spatial symmetry of the incident photons. Tentatively, self-trapped high state symmetrical photons oscillate in unison with electron vortices supporting formation of the standing wave in synchronized water. The open circle itself is only one of two parts in the resonance form of hydrogen. Therefore, there is no complete reformation or reconstruction of an ordinary standing wave form with the open circle only when exposed to light. With the open circle or geometrical proportions different from Fibonacci numbers the observed increase in fractality of conditioned light is insufficient to make up a self-regulative configuration of synchronized water. Accordingly, the complete SS geometry is needed to make a significant conformational change in the outcome of ordinary water.

### Example 1

### Water Synchronization Test

The density, the dielectric constant (permittivity), the surface tension and the temperature profile at the freezing and melting point were examined in synchronized distilled water. Distilled water (1 litre stored in a transparent plastic bottle, Apoteket AB, Sweden) was exposed to daylight and a TGM (e.g. SS imprinted on laser transparency film (3M)) for 24 h at ambient temperature (22-23° C.). The TGM used was an SS matrix having a closed small circle (see FIG. 1a) and an SSc matrix having an open small circle (see FIG. 1b), both having an outermost circle diameter of 13 mm and an innermost circle diameter of 1 mm. Each of the matrixes were connected on the plastic bottle. The line width of the outermost circle was 0.07 mm. The temperature characteristics were followed within NiCrNi sensors via collection (Temperaturlogger, Nordtec AB, Sweden) every third second during 8 h of temperature data in control and synchronized distilled water, respectively, wherein the water samples were stored in a freezer at −18° C. The NiCrNi sensors were tightly connected at the center of 50 ml glass tubes (4 cm below the water surface), which were filled up with 45 ml control or synchronized distilled water. The density of the synchronized water was analysed by balancing (Mettler, GTF, Sweden) in a known volume of water. The melting point was determined from the frozen glass tubes when stored at ambient temperature (22-23° C.). The dielectric constant of water was analyzed with a Percometer (Adek Ltd, Estonia). The dielectric probe was shielded in a Faraday's cage.

The properties of synchronized water after the TGM conditioning are listed in Table 1 below.

TABLE 1
The characterization is related to conditioning of distilled water for 24
h with either of the following TGM (SS, SSC) and exposed for daylight.

| | Matrix | | |
|---|---|---|---|
| Parameter | Reference | SS | SSc |
| Density (g/ml) # | 0.997800 | 0.998246 ± 0.000265*** | 0.998133 ± 0.000278** |
| Permittivity, Faraday box | 80 (77.7\hat{ }) | 82.50 ± 0.27* | — |
| ((F/m) # | | | |
| Water temperature at the | 0 | −6.9 ± 1.7*** | −8.2 ± 0.3*** |
| freezing point (° C.) [symbol] | | | |
| Melting point (° C.) [symbol] | 0 | 0.2 ± 0.027* | 0.1 ± 0.00* |
| Surface Tension (dyn/cm) # | 73 (72.9\hat{ }) | 72.3 ± 0.054*** | 72.7 ± 0.02** |

[symbol]

All values are the mean (±SD) of two repetitive analyses during three consequtive days.
# Values are the mean of thirty repetitive measurements analysed on three occations at 22° C.
\hat{ }Experimental reference value.
*P < 0.05;
**P < 0.01;
***P < 0.01 [symbol]
indicates data missing or illegible when filed

Thus, the criterion for synchronized water is the following:

Water in a distilled condition and at atmospheric pressure which, with the basis of the results obtained for the SS and SSc matrix, respectively, has

a) a density of from 0.997855 g/ml to 0.998511 g/ml at 22° C.,

b) a water temperature from −8.5° C. to −5.2° C. at the freezing point,

c) a melting point from 0.1° C. to 0.2° C.,

d) a surface tension from 72.3 dyn/cm to 72.7 dyn/cm at 22° C., and

e) a dielectric constant from 82.23 F/m to 82.77 F/m.

The synchronized water was found to have a substantially higher density at ambient temperature (22° C.) after TGM treatment. The relative density calculated on the basis of the average of measured densities after TGM condition varies between 0.997855 g/ml and 0.998511 g/ml (P<0.01-0.01).

The average water temperature at the freezing point in synchronized water varies between −5.7° C. and −8.2° C. (P<0.001), between −8.6° C. and −3.7° C. with the basis on the standard deviation. The corresponding melting point range was 0.1-0.2=C (P<0.05). The dielectric constant during the TGM treatment was substantially increased and was 82.5 F/m, between 82.23 F/m and 82.77 F/m (P<0.001) with the basis on the standard deviation. The surface tension was substantially reduced after the TGM treatment, in particular with the SS matrix (72.3 dyn/cm (P<0.001)) and the average was in the range of 72.25-72.72 dyn/cm.

Further experiments with SS matrixes having different outer circle diameters and fixed or varying inner circle diameters have led to temperatures at the freezing point of higher than the value −8.6° C. in the interval above and up to −3.6° C.±1.2, i.e. −2.4° C. at most:

| | mean | sd |
|---|---|---|
| SS total | −7.1 | 0.7 |
| SS YD 5 mm | −6.8 | 0.6 |
| SS YD 13 mm | −6.9 | 1.7 |
| SS YD 21 mm | −6.2 | 2 |
| SS 5 variable ratio | −4.7 | 1.9 |
| SS 13 variable ratio | −4.8 | 1.3 |
| SS 21 variable ratio | −3.6 | 1.2 |
| Total variation | −5.7 | 1.3 |

Thus, a water could be regarded as being synchronized according to the present disclosure if it shows a temperature at the freezing point ranging from −9.0° C. to −1.0° C., such as from −8.68° C. to −5.2° C., if treated and measured in accordance with Example 1. This parameter is sufficient to measure with a view to establishing whether the water is synchronized or not.

Further, said synchronized water also have the properties defined under a), and c)-e) above. Thus, if the water also has

a) a density of 0.997830 g/ml to 0.9990 g/ml at 22° C.

c) a melting point of 0.05° C. to 0.25° C.

d) a surface tension of 72.0 dyn/cm to 72.8 dyn/cm at 22° C., and

e) a dielectric constant of 82.0 F/m to 83.0 F/m₁₁

it can be regarded as synchronized according to the present disclosure.

The observed temperature characteristics of synchronized water is only to be considered anomaly in relation to ordinary stagnant water, but highly normal according to moving, vortex, interfacial, exclusion zone and super cooled water. Opposed to ordinary stagnant water synchronized water is an active oscillatory medium based on transformation of low-density into high-density energy, i.e. thermodynamic electron excitation energy and/or emission of spectral light.

The reason why slightly different values for the synchronized water were obtained during use of different matrixes is that the organization of water molecules is uniquely, specifically and selectively connected in a field-like structure due to the fact that each geometry creates a selective interference pattern with incident spectral light, for example, light in the red and low infrared part of the light spectrum, wherein formations arise in the water which manifest in a unique profile as to physical and chemical parameters, thus somewhat deviating between different matrixes. Each TGM represents a unique resonance structure, i.e. a standing wave pattern, of a corresponding atom or molecule, where the SS geometry is equivalent to the resonance form of molecular hydrogen. The spectral and highly organized light is completely absorbed by water molecular hydrogen bondings probably affecting and synchronizing the spin rotation of hydrogen atoms in water. The absorption of spectral light is equivalent to the emission wavelengths of a particular reactive oxygen species (ROS), namely singlet oxygen, which indicates that a macroscopic spin-spin coupling of the hydrogen atoms in synchronized water depends on formation of ROS and specifically singlet oxygen.

The increase in permittivity indicates that the strength and extent of the hydrogen bonding both increase, which makes the water act as a unified system. This 1) makes the mobility of the molecular water dipole difficult and restricts the ability of the water molecule to oscillate at a higher frequency, 2) increases the inertia in the rotation of the water molecules, i.e. increases the friction and thus the dielectric loss, and 3) changes the ordinary water structure.

The reduction of the water temperature to below zero before the freezing takes place favours the formation of a system organizing water structures stabilized by the hydrogen bonds. A change of the morphology in synchronized water requires time before the shift to the hexagonal network configuration at the freezing point. The marginally higher melting point of the synchronized water in the form of ice indicates re-formation of the self-organizing water structures at a temperature above the melting point of ordinary control water.

The reduction in surface tension makes the synchronized water more wet and more fluid, which favours the co-operativity and the adjacent dynamic mobility and the fluidality in the close adaptability between neighbouring water structure modules, i.e. a condition which increases the water density.

Moreover, the difference due to the conditioning between synchronized and control water is so pronounced that there is no doubt as to whether water is synchronized or not. All differences measured in the parameters described are profiled and changed in a coherent way, which nevertheless is unique for each resonance form. Thus, this synchronization test is indicative of whether a TGM has the ability to synchronize water or not, i.e. if the parameter values measured in this test lie within the critical parameter ranges. If so, the TGM too is considered to reduce or eliminate tinnitus of a patient by synchronization of the water in the patient's skin and body fluids.

To conclude, the result indicates that in distilled synchronized water the binding and the order of water structures are fractal, highly organized with field-like properties. The density increase indicates formation of a “fluidal crystal structure” which differs from the ordinary hexagon-like structure present in cold water and ice. As the field-like structural configuration observed during the freezing experiment is regained as the temperature increases above the zero-level, which differs from the ordinary hexagonal structural ordering, the synchronized water with a self-regulating inherent organization favours a molecular feedback formation of tetrahedal molecular structures having a high structural symmetry regulated by self-identical hydrogen bonds. The fractal organization in synchronized water makes up a highly stable inter- and intra-molecular self-organizing biosystem.

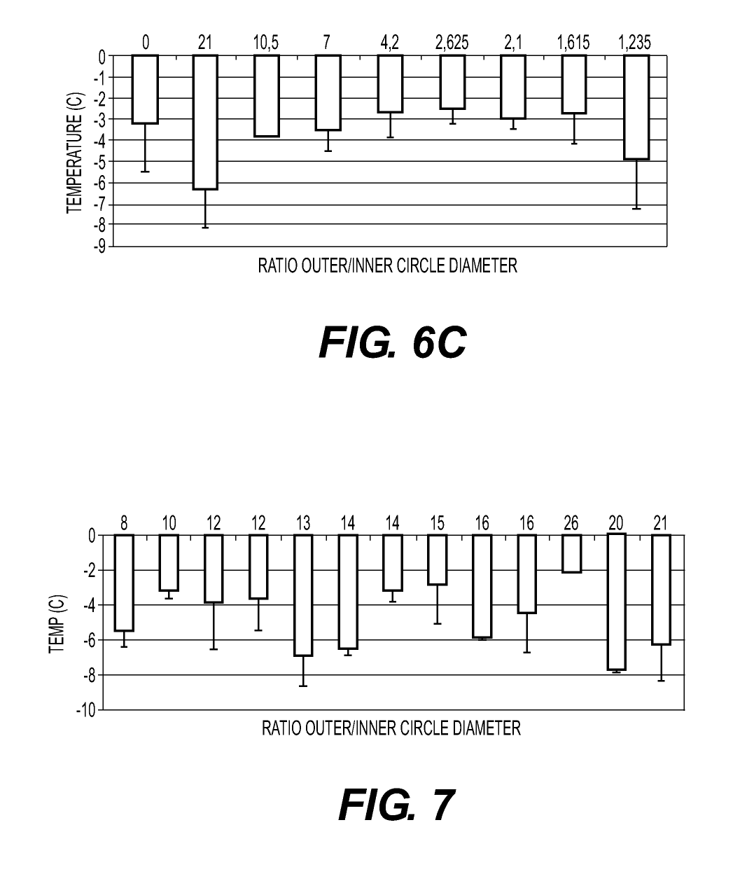

Similar results as above, although not presented here, have been obtained for an SS matrix having an outermost circle diameter of 5 mm and 21 mm, respectively, and an innermost circle diameter of 1 mm (see FIGS. 6a-6c).

### Example 2

### Tinnitus Treatment Test

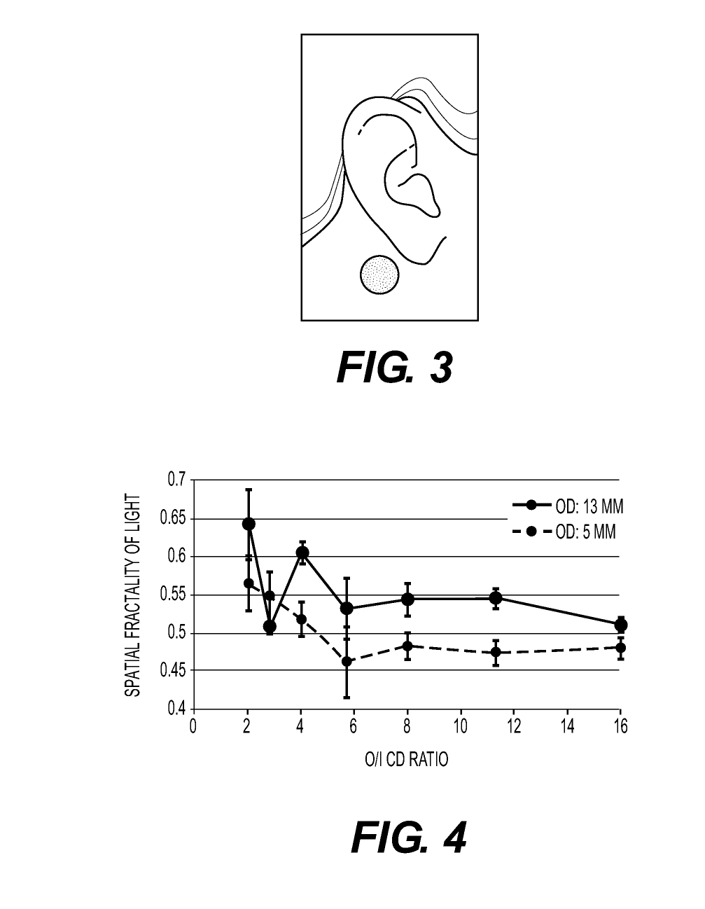

A pilot study was performed on 20 subjects suffering from undiagnosed, i.e. not clinically manifested, tinnitus with various severities in the experience of unpleasant and painful tinnitus sensations. The topographic geometrical matrix was applied to a transparent plaster with a skin friendly adhesive locally at the cranial base behind the ear associated with the tinnitus symptoms (see FIG. 3).

The plaster used in this example having a topographic geometrical matrix imprinted thereupon is MACal® 8199, which is commercially available. The backing paper for this plaster was MACal 819 n.t.c., mp312, Century 8 White, also commercially available, and this backing paper has to be released from the bottom side of the plaster before adhering the plaster to the patient's skin.

The cover colour of the plaster was LITHOCURE 3G™ (U03XXXXX). Detailed information about this cover colour may be obtained from Flint Group Narrow Web, P.O. Box 1003, 231 25 Trelleborg, Sweden, www.narrowweb@flintgrp.com, or from Flink Group Sweden AB, XSYS Print Solutions, having the same address.

For most of the treated patients the topographic geometrical matrix on the plaster had the form of an SS matrix (see FIG. 1a), and the outer circle having a diameter of 13 mm and the dosed inner circle having a diameter of 1 mm were imprinted on the plaster with the silver colour 6182c Conductive Dry Offset inc, supplied from Luminescence Incorporated, The Fairway, Bushfair, Harlow, Essex, CM18 6NG, UK. For the rest of the patients the topographic geometrical matrix on the plaster had the form of the flower of life, as shown to the right in FIG. 2, wherein each circle had a diameter of 13 mm and the smaller closed dot formed by other circle arcs in the center of each circle had a diameter of 1 mm. This matrix was imprinted on the plaster in the same way as the SS matrix.

This SS based device used for treatment of tinnitus is also called Aklo Plaster “SS Microblue”.

Hypothetically, the circular polyester plaster supporting the matrixes and having an outer diameter of 27 mm and a thickness of 100 μm affects body water by giving ordinary daylight altered and highly organized qualities which have the ability to influence the body water so that it becomes fractal and synchronized in accordance with the qualities of ordinary synchronized liquid water. All subjects had been suffering from complaints and pain for several years and experienced substantial problems classified as moderate tinnitus before the treatment. The result of the treatment was as follows;

TABLE 2

| Test subject | Symptoms, treatment time and outcome |
|---|---|
| Woman, | Problems disappeared within 1 week. |
| 55-60 years | |
| Woman, | Problems disappeared within 24 hours, very substantial |
| 55-60 years | improvement. |
| Woman, | Tinnitus sensations for two years. Constant low-level |
| 40 years | pulsating noise exacerbated by running water or rustling |
| | plastic bags to the point where those noises were almost |
| | unbearable. By two weeks most background interference |
| | subsided. After another two weeks she hardly notices any |
| | disturbances and it is not a problem for her. |
| Woman, | Tinnitus sensations for 6 months. Symptoms subside within |
| 59 years | two weeks. |
| Woman, | More or less tinnitus symptoms for 10 years. Headache and |
| 49 years | tinnitus sensation located central in the head moved to |
| | the left ear and became very weak. |
| Woman, | Severe tinnitus. Tone gone after a week. After another two |
| 62 years | weeks all sounds gone. |
| Woman, | Noise since 10 years. After one week the tone was gone. |
| 44 years | |
| Woman, | Severe tinnitus. After 5-6 h the sound declined. No tinnitus |
| 60 years | for months. |
| Woman, | Severe tinnitus and nerve damage. Effect after 48 h. |
| 58 years | |
| Woman, | Stress induced tinnitus. Mild change during day-time and |
| 58 years | huge change during the night. Sleep improved. |
| Woman, | Whiplash. Huge change, no tone and sound after a few |
| 39 years | days. |
| Woman, | Loud noise from cell phone. After one night the tone |
| 32 years | disappeared. |
| Woman, | Dizziness. Menier's disease, direct effect. |
| 82 years | |
| Woman, | Heavy tinnitus in both ears since more than 10 years. After |
| 43 years | about two hours following the treatment she had the feeling |
| | as if frequencies would get changed (like it happens on a |
| | shortwave-radio), when shifting frequencies for about three |
| | to four minutes. Thereafter the tinnitus was reduced |
| | considerably. The disruption by the tinnitus seemed to have |
| | lost its aggression completely, As of today, the tinnitus is |
| | still there, but not to be compared with prior-to-the-patch, |
| | it is much weaker. And it stayed weaker. It has not yet |
| | disappeared completely, yet. |
| Man, | Substantial stress. Problems relating to both signal and pitch |
| 55-60 years | disappeared within 24 hours. Travelled abroad (by |
| | airplane). Was exposed to a high sound level on a nightclub |
| | and at a Formula 1 race. The tinnitus then returned. Treated |
| | again, and once more the problem disappeared within 24 |
| | hours. |
| Man, | Suffers from tinnitus for 15 years. The problems relating to |
| 45 years | signal sound disappeared within 24 hours and periodically |
| | also the problems relating to the buzz. |
| Man, | Acute tinnitus, loud noise for several hours. Problem |
| 60 years | disappeared within 24 hours. Complete recovery. |
| Man, | Swimmers ear. Tone gone. Can still hear a light sound but |
| 45 years | no tone. |
| Man, | Stress induced tinnitus. Tone gone in 24 h, came back gone |
| 56 years | again. |
| Man, | Loud war noise. Mild change during day-time and huge |
| 76 years | change during the night. Sleep improved. |
| Man, | Hunters ear. Four days of slight difference. |
| 76 years | |
| Man, | Severe hunters ear. A couple of days with placebo effect? |
| 81 years | |
| Man, | Loud music induced tinnitus. No difference to start, then |
| 33 years | the signal went and came back and so on. |
| Man, | Hunters ear. Signal gone after 24 h. |
| 46 years | |
| Man, | Physical damage. Off and on came back after a few days. |
| 52 years | |
| Man, | High sound. Tone gone after a few days. |
| 35 years | |

Our clinical results recognize that the device according to the present disclosure applied on the skin close to the affected ear is in most cases highly effective in giving more or less persistent relief from tinnitus sensations. Despite a broad range in the experience of tinnitus sensations in terms of the time of suffering and the severity of symptoms among the test subjects, the applied clinical protocol using a continuous application of the plaster until symptoms subsided had long-term beneficial effects on tinnitus by restoring the hearing function. Since the plaster was applied on a non-auditory structure area on the skin behind the ear, the effect is considered mediated by a non-classical somatosensory pathway related to a cellular and body water-inducible stimuli that can change the molecular structure of water in order to achieve sympathetic/parasympathetic balance in the ANS, leading to physiological homeostasis diminishing or eliminating tinnitus sensations. Preliminary short-term 10 minutes electrocardiography (ECG) obtained in three of the 26 tinnitus patients showed a significant decrease in two PSD parameters, normalized low frequencies (LFnorm) and the low frequency/high frequency (LF/HF) ratio without affecting heart rate. These data indicate an involuntary shift in ANS activity from a sympathetic load towards a tendency of parasympathetic regulation, thus, reducing physiological arousal and environmental induced stress in these individuals.

In FIGS. 4, 5, 6a-8c, and 7 shown and disclosed here, the staples represent the temperature measured at the freezing point, and the vertical line below each staple represents the error bar corresponding to the standard deviation.

### Example 3

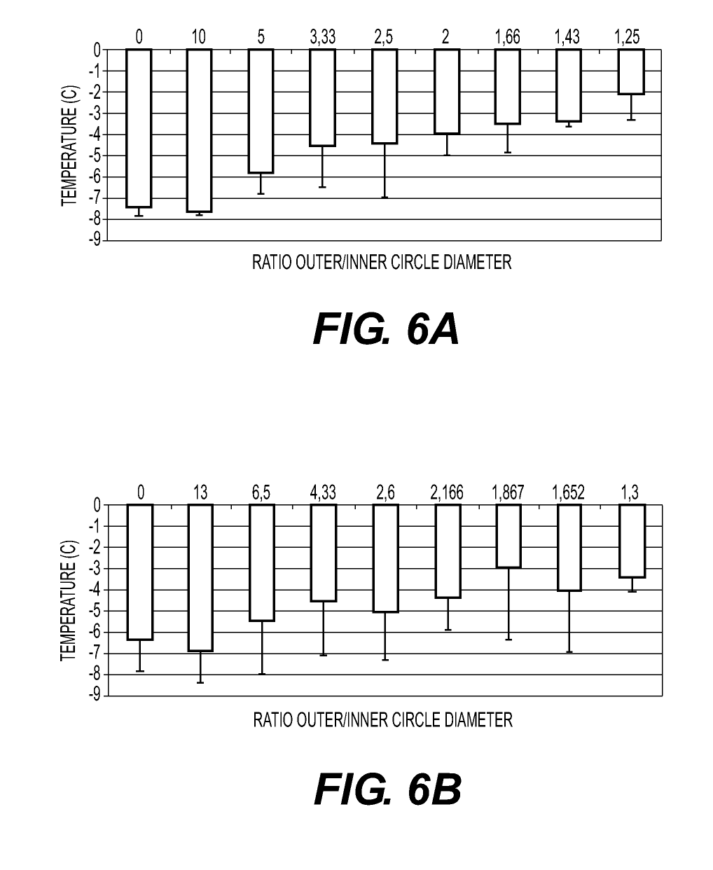

FIG. 5 below shows freezing point measurement experiments performed for 8 different embodiments of the TGM according to the present disclosure.

The matrixes 1-8 tested correspond to the matrixes 1a)-h) in FIG. 1 in the following way:

### Matrix 1=1a)

### Matrix 2=b)

### Matrix 3=1e)

### Matrix 4=1f)

### Matrix 5=1d)

### Matrix 6=1e)

### Matrix 7=1g)

### Matrix 8=1h)

For the matrixes 1-8 the outer diameter was 13 mm, and the inner diameter 1 mm. For the matrixes 5-8 the diameter of the intermediate circle was 6.5 mm. The freezing point measurements were performed as disclosed in Example 1. As appears from FIG. 5, all of the tested matrixes 1-8 showed a temperature at the freezing point of at most about −3° C. and are therefore effective when used in connection with the present disclosure, i.e. give rise to synchronized water per definition. The SS matrix (1) gave the lowest temperature at the freezing point, followed by matrix 4.

### Example 4

FIGS. 6a-6c show results of temperature measurements at the freezing point, performed as defined in Example 1, for an SS matrix having an outer circle diameter of 5 mm and various inner circle diameters (FIG. 6a) from 0 mm up to 4 mm with increments of 0.5 mm, for an SS matrix having an outer circle diameter of 13 mm and various inner circle diameters (0 mm, 1 mm, 2 mm, 3 mm, 5 mm, 6 mm, 7 mm, 8 mm, and 10 mm, respectively) (FIG. 6b), and for an SS matrix having an outer circle diameter of 21 mm and varying inner circle diameters (FIG. 6c).

The numbers shown in each staple represent the ratio between the outer circle diameter and the inner circle diameter for each matrix.

The lowest freezing points were observed at a ratio between the outer circle diameter and the inner circle diameter corresponding to ratios between numbers in Fibonacci's sequence of numbers, or closely corresponding (within ±10%), such as the ratios 10/0.5 (almost corresponding to 21/2) in FIG. 6a, 13/1 in FIG. 6b, and 21/1 in FIG. 6c. It could also be noted that for a given outer circle diameter, increasing inner circle diameters, i.e. gradually reduced ratios, generally, with a few exceptions, give rise to less temperature decreases at the freezing point. It should be noted that according to FIGS. 6a-6c a matrix in the form of a circle having a diameter of 6 mm, 13 mm, and 21 mm also has been shown to give a substantially reduced temperature at the freezing point in synchronized water. However, although this matrix fulfils the definition of synchronized water disclosed above, it gave no effect at all when used in the method for treatment of tinnitus, according to the present disclosure.

### Example 5

FIG. 7 shows results of temperature measurements at the freezing point, performed as defined in Example 1, for an SS matrix having an inner circle diameter of either 0.5 mm (staple numbers 1, 3, 6, 9, 11, and 12 from the left) or 1 mm (the rest of the staples) and having a varying outer circle diameter. The lowest temperatures at the freezing points are observed for the ratios 13/1 and 21/1, which both correspond to Fibonacci's sequence of numbers.

The present disclosure has been described above with reference to various embodiments of the disclosure. However, a skilled person in the art realizes that further variants within the scope defined by the present claims are intended to be included in the present disclosure.

### LITERATURE

1. Holgers K M. “Mechanisms and classification oftinnitus—a discussion paper” Audiological Medicine, 2003a; 1; 1-4.
2. Möller A R. Tinnitus: presence and future. Prog Brain Res 2007; 166; 3-16.
3. Axelsson A, Ringdahl A. Tinnitus—a study of its prevalence and characteristics. Brit J Audiol 1989; 23; 53-62.
4. Davis, A C. (1995) Hearing in adults, London, UK.
5. Johansson M S, Arlinger S D. Prevalence of hearing Impairment in a population in Sweden. Int J Audiol 2000; 40(1); 18-28.
6. Holgers K M, Erlandsson S I, Barrenas M L. Predictive factors for the severity of tinnitus. Audiol 2000; 39; 284-91.
7. Holgers K M. The prevalence of tinnitus in 7 year-old schoolchildren. Eur J Pediatr 2003b; 162; 276-8.
8. Holgers K M, Erlandsson S I, Barrenas M L. Predictive factors for the severity of tinnitus. Audiol. 2000, 39(5):284-91.
9. Gangi S, Johansson O. Skin changes in screen dermatitis versus classical UV and ionising radiation-related damage-similarities and differences. Experiment Dermatol 1997; 6; 283-291.
10. Kaplan S, Kaplan R. The experience of nature: Psychological experience. New York: Cambridge university press, 1989.
11. Möller A R. Tinnitus and pain. Prog Brain Res 2007; 166; 47-53.
12. Ridder D D, Van de Heyning P. The Darwinian plasticity hypothesis for Tinnitus and pain. Prog Brain Res 2007; 166; 55-59.
13. Ulrich R S. Aesthetic and affective response to natural environment. In. Altman I, Wholwill J F (eds), Behavior and the natural environment. New York, Plenum. 1983; 6; 85-125.
14. Johansson B. Synchronized water and production thereof. Patent application, PCT/SE2008/000119.
15. Johansson B. Effects of functional water on heart rate, heart rate variability and salivary IgA in healthy humans—A pilot study. J Altem Compl Med 2009; 15; 871-877.
16. Johansson B, Kronholm J. Effects of functional synchronized water on heart rate variability, fractal heart rhythm dynamics and salivary IgA in healthy subjects—a pilot study. Manuscript in preparation.
17. Johansson B. Heart rate and heart rate variability response to the transpiration of vortex-water by Begonia Eliator plants to the air in an office during visual display terminal work. J Altern Compl Med 2008; 14; 993-1003.
18. Goldberger at al. Fractal dynamics in physiology: Alterations with disease and aging. PNAS 2002; 99; 2466-2472.
19. Uu K, CruZan D, and Saykally R J. Water clusteras. Science 271, 929-933, 1996.
20. Tsal C J, and Jordan K D. Theoretical study of small water clusters. J Phys Chem 97, 5208-5210, 1993.
21. Lo S Y, U W C and huang S H. Water dusters in life. Med Hypothesis 54, 948-53, 2000.
22. Teschke o and de Souza E F. Water molecule dusters measured at water/air interfaces using atomic force microscopy. Phys Chem Chem Phys 7, 3856-3865, 2005.
23. Burenkov N A. The role of system-forming modular water structures in self-organization of biological systems. J Molec Liquids 106, 257-275, 2003.
24. Diplock A T, Aggret P J. Scientific concepts of functional foods in Europe. Consensus document. Br J Nutr 81, Suppl 1:51-527, 1999.
25. Del Giudice E, Tedeschi A. Water and autocatalysis in living matter.
Electromagn Biol Med. 2009, 28, 46-52.
26. I Prigogines, I Stengers. Order out of Chaos. Bantam Books, 1984.
27. Fresnel zone plate: http://en.wikipedia.org/wiki/Fresnel_zone_plate.
28. M Chown. Our world may be a giant hologram. New Scientist 2009, 2691, 24-27.

1. A device for treatment of tinnitus comprising at least one topographic geometrical matrix and a support, wherein each topographic geometrical matrix is applied to the support and comprises
a) at least two concentric circles,
b) a circle having at least one circle inscribed, wherein said circle and said at least one circle have a common tangential arc point;
wherein for both a) and b) above the innermost circle is open or closed, the diameter of the outermost circle is at most 130 mm, and the diameter of the innermost circle is at most 3 mm, and wherein the ratio between the diameter of the outermost circle and the diameter of the next circle is at least 1.3;
c) a set of identical circles arranged with the basis on the pattern of the flower of life,
and/or asymmetrical variants of a)-c),
wherein the ratio between the diameter of the outermost circle and the diameter of the next circle counted inwards toward the common center of the circles in each topographic geometrical matrix is the ratio between any number being at least 2 in Fibonacci's sequence of numbers and any number lower than that being at least 1 in said Fibonacci's sequence of numbers, including ±10% for each ratio.

2. (canceled)

3. The device according to claim 1, wherein the topographic geometrical matrix is formed of two concentric circles, the innermost circle being closed (an SS matrix).

4. The device according to claim 3, wherein the relationship between the outermost circle and the innermost circle of the matrix is 13±10%.

5. The device according to claim 1, wherein the topographic geometrical matrix according to claim 1 a) and b) has a diameter of the outermost circle ranging from 2 mm to 130 mm, and a diameter of the innermost circle ranging from 0.5 mm to 3 mm.

6. The device according to claim 1, wherein one or more rings formed between circles in a matrix containing several concentric circles are closed.

7. The device according to claim 1, wherein the line width of the circles of the topographic geometrical matrix ranges from 0.05 mm to 2.0 mm.

8. The device according to claim 1, wherein the topographic geometrical matrix is fully or partially colored with one or more metallic colors.

9. The device according to claim 1, wherein the topographic geometrical matrix comprises a metal.

10. The device according to claim 1, wherein the topographic geometrical matrix is plated, imprinted, etched, glued, or laminated on the support.

11. The device according to claim 1, wherein the support comprises glass, cardboard, paper, plastic, sheet metal, or a natural material.

12. The device according to claim 11, wherein the support adheres to human or animal skin.

13. The device according to claim 1, wherein the support is a plaster having an upper side provided with the topographic geometrical matrix and a bottom side that adheres to human skin.

14. The device according to claim 13, wherein the topographic geometrical matrix is imprinted with silver colour on the plaster.

15. The device according to claim 11, wherein, before use, the support is provided on its adhesive bottom side with a peelable backing paper.

16. The device according to claim 1, wherein several identical or different topographic geometrical matrixes are applied to the support.

17. A method for therapeutic treatment of tinnitus comprising applying at least one device according to claim 1 on the body of a tinnitus patient and subjecting the at least one device to light with a wavelength of 360-4000 nm.

18. A method according to claim 17, wherein the patient is subjected to treatment with said device for up to one month.

19. The method according to claim 17, wherein the tinnitus is mild tinnitus, moderate tinnitus, or severe tinnitus.

20. A method for therapeutic treatment of tinnitus comprising administering orally to a tinnitus patient water or a water-containing medium which has been subjected to light that has passed at least one topographic geometrical matrix according to claim 1.

21. The method according to claim 20, wherein the treatment period is for up to one month.

22. The device according to claim 5, wherein the diameter of the outermost circle is at most 80 mm.

23. The device according to claim 5, wherein the diameter of the outermost circle is at most 30 mm.

24. The device according to claim 7, wherein the line width of the circles of the topographic geometrical matrix ranges from 0.1 mm to 1.5 mm.

25. The device according to claim 7, wherein the line width of the circles of the topographic geometrical matrix ranges from 0.5 mm to 1.0 mm.

26. The device according to claim 9, wherein the metal is chosen from copper and brass.

27. The device according to claim 12, wherein the support adheres to human or animal skin by means of an adhesive surface.

28. The device according to claim 13, wherein the plaster is transparent.

29. The method according to claim 17, wherein the device is applied in the vicinity of the affected ear.

30. The method according to claim 17, wherein the light is daylight.
